(19) United States
(12) Patent Application Publication
(10) Pub. No.: US 2004/0242862 A1
(43) Pub. Date: Dec. 2, 2004

(54) Method for producing a cellulose ether of low viscosity by means of acid oxidative decomposition of ground and dried cellulose ethers

(76) Inventor: Alf Hammes, Huenstetten-Goersroth (DE)

Correspondence Address: Richard S Roberts, Roberts & Roberts, PO Box 484, Pinceton, NJ, 08542, US

(21) Appl. No.: 10/487,958

(22) PCT Filed: Aug. 21, 2002
(86) PCT No.: PCT/EP02/09319

(30) Foreign Application Priority Data
Aug. 25, 2001 (DE) 10141680.6

(51) Int. Cl. C08B 11/20; C07H 15/04

(52) U.S. Cl. 536/120; 536/85

(57) ABSTRACT

A method for the depolymerization of cellulose ethers by acid oxidative decomposition. Ground and dried cellulose ether is exposed to gaseous acid or is sprayed with an acid solution. It is brought into contact with an oxidizing agent or an oxidizing agent solution, and depolymerized at temperatures in the range from 50 to 120° C. over a period in the range from 0.01 to 10 hours. The acid is subsequently neutralized by adding a base. The water content of the reaction mixture does not exceed 10% by weight during depolymerization.

The present invention relates to a method for producing cellulose ethers of low viscosity by depolymerization by means of acid oxidative decomposition of ground and dried, i.e. fully processed, cellulose ethers of a higher degree of polymerization.

The decomposition of cellulose ethers with high degrees of polymerization has been known for a long time and can be achieved in diverse ways. Decomposition to products of very low viscosity in particular has attracted great attention because these products can be employed advantageously inter alia as coating material for active pharmaceutical ingredients or seeds, but also for example as protective colloid in emulsion polymerization. Products of very low viscosity referred to hereinafter are cellulose ethers whose Höppler viscosity measured at a concentration of 2% (absolutely dry) in water at 20° C. is ≦50 mPas.

The methods employed to decompose cellulose ethers include, besides acid-catalyzed hydrolytic cleavage of the acetal linkage, inter alia oxidative decomposition and decomposition by high-energy radiation or micro-organisms/enzymes.

Methods for the oxidative decomposition of cellulose ethers are described inter alia in U.S. Pat. No. 2,912,431, U.S. Pat. No. 4,316,982, CH-B-461 455, DE-A-20 16 203, GB-B-953 944 and DE-A-198 54 770.

U.S. Pat. No. 2,912,431 describes a method in which hypohalites, peroxides or periodates decompose carboxymethylcelluloses in a mixture with aqueous alcohol at 40 to 80°C. with simultaneous bleaching.

The decomposition of water-moist cellulose ethers with a dry content of 40 to 75% by weight with ozone/air/oxygen mixtures at 0 to 60° C. is described in U.S. Pat. No. 4,316,982.

CH-B-461 455 describes a method in which the cellulose ether with a maximum water content of 75% by weight is mixed with 0.1 to 10% by weight aqueous hydrogen peroxide solution. The resulting mixture is then oxidatively decomposed and dried at 100 to 250° C. until the H₂O₂ is consumed.

DE-A-20 16 203 describes a method for the decomposition of cellulose ethers in which a substantially dry powder with a maximum water content of 5% by weight is mixed with a hydrogen peroxide solution and decomposed at 50 to 150° C.

In GB-B-953 944, the viscosity of water-soluble, nonionic cellulose ethers is reduced in the dry or moist state by reaction with H₂O₂ at elevated temperatures.

DE-A-198 54 770 describes a method for the depolymerization of moist cellulose ethers at temperatures in the range from 60 to 125° C. by spraying with a hydrogen peroxide solution.

However, oxidative decomposition of cellulose ethers usually leads, because of the comparatively large amount of oxidizing agent used or, alternatively, disproportionately long reaction times with nonselective chain cleavage, to the formation of numerous byproducts, including oxidized ones, which reduce the purity of the product.

Simple hydrolytic decomposition methods, which are neutral in relation to functional groups, with inorganic or organic acids are described for example in UA-A-1 679 943, U.S. Pat. No. 1,943,461, EP-A-0 497 985 and EP-A-0 210 917.

In U.S. Pat. No. 1,943,461, the preground cellulose ethers are decomposed with a multiple of their weight of dilute acids or mixtures thereof (concentration: 0.5 to 5% by weight) in a closed pressure vessel under a pressure of 0.7 to 5.2 bar and at temperatures of 115 to 160° C. for 20 to 60 minutes. U.S. Pat. No. 1,679,943 describes the decomposition of cellulose ethers with various acid mixtures, with no pressure vessel or elevated temperature being required. However, the resulting reaction times are very long, especially at room temperature, and may be in the region of several days.

In EP-A-0 497 985, pulps with a low copper number are converted into cellulose ethers, and the latter are washed, dried, ground and mixed at a temperature of about 70° C. with a 0.5% by weight aqueous HCl solution. The resulting cellulose ethers have very low viscosities (<20 mPas, concentration 2.0% at 20° C.).

A similar method is described in EP-A-0 210 917. In this case, a cellulose ether powder containing 3 to 8% by weight water is decomposed with 0.1 to 1% by weight of an aqueous HCl solution at 40 to 85° C.

Decomposition to products of very low viscosity in particular can also be achieved by using HCl as gas. Methods of this type are described for example in U.S. Pat. No. 3,391,135, U.S. Pat. No. 4,061,859 and WO 00/32637.

UA-A-3 391 135 discloses a method for producing cellulose ethers with solution viscosities of less than 10 mPas (concentration 2.0% at 20° C.) from cellulose ether powders of higher viscosity and water contents below 5% by weight at 30 to 80° C. Excess HCl gas is removed and the cellulose ether is then neutralized by admixing a weak base.

In U.S. Pat. No. 4,061,859, cellulose ethers are decomposed as dry powders with a water content of 0.01 to 5% by weight with hydrogen halide at 15 to 80° C. and then neutralized by admixing sodium bicarbonate or passing in gaseous ammonia. The material obtained is bleached with sulfur dioxide gas with which the decomposed material is brought into contact after the depolymerization stage. It is possible by this method to decompose cellulose ethers to products of very low viscosity from an initial viscosity of several hundred thousand mPas. The bleaching stage following the depolymerization makes it possible to lighten the color of the products but means an additional method step. In WO 00/32637, cellulose ethers are, with the aim of depolymerization, brought into contact with acids while agitating continuously at 50 to 130° C.

Hydrolytic decomposition is neutral in relation to functional groups and can be employed to produce products of very low viscosity. However, general problems are the color of the products, and the formation of brownish-black lumps of product. The latter are formed in particular due to non-uniform distribution of the water and the concentration of the acid in conglutinated regions with a high water content. A subsequent bleaching step is often necessary in order to obtain uncolored products. In this case, either oxidizing agents are employed in amounts which significantly increase the content of oxidized product constituents, or new byproducts are additionally formed due to the introduction of nitrogen- or sulfur-containing compounds.

A combination of acid hydrolytic and oxidative decomposition in a concentrated aqueous slurry is described in DE-A-199 41 893. In this case, an excess of water is used in a two-phase system (solid/liquid). The maximum ratio in parts by weight of water (slurry medium) to cellulose ether is, however, 10:1. This method results in substantially uniform, low-salt cellulose ethers with a high degree of whiteness, although with a reduced yield of about 80 to 96%. Subsequent removal of the depolymerized cellulose ether from the slurry medium, and the necessary drying and grinding of the decomposed material are very difficult because of the high water-retention capacity, the high thermal plasticity and the great tackiness of the depolymerized products. It was therefore an object of the present invention to provide a method for the depolymerization of cellulose ethers which does not have the prior art disadvantages mentioned. In particular, possible ways were sought for producing cellulose ethers of very low viscosity (≦50 mpas) which, besides minimal amounts of oxidized constituents, have a high degree of whiteness from cellulose ethers of higher viscosity (>50 mPas up to several 100 000 mpas) in quantitative yield. It was further intended to avoid the problems of drying and grinding associated with products of very low viscosity.

This object is achieved according to the invention by a method for the depolymerization of cellulose ethers by acid oxidative decomposition, which is characterized in that ground and dried cellulose ether is exposed to gaseous acid or is sprayed with a solution of an acid, and is brought into contact with an oxidizing agent or a solution of an oxidizing agent, in that it is depolymerized at temperatures in the range from 50 to 120° C. over a period in the range from 0.01 to 10 hours, and subsequently the acid is neutralized by adding a base, it not being permissible for the water content of the reaction mixture to exceed 10% by weight during the depolymerization.

It has surprisingly been found that the degrees of whiteness achieved by combining acidic and oxidative decomposition of cellulose ether powders with a water content of less than 10% by weight cannot be attained without the simultaneous use of acidic and oxidizing reagents. Besides efficient depolymerization with low acid input, the combination with small amounts of an oxidizing agent leads to an increase in the degree of whiteness of the depolymerized products while limiting the content of oxidized additional constituents.

Since the decomposition is carried out on fully processed cellulose ether with a water content of less than 10% by weight, subsequent drying and grinding/sieving of the material is unnecessary. The depolymerized cellulose ether is obtained in quantitative yield. The advantage compared with the method described in DE-A-1 99 41 893 is thus in particular the quantitative yield of depolymerized product, the avoidance of contaminated waste water, the distinctly reduced acid input, and the dispensing with the problematic drying and grinding of the depolymerized cellulose ether.

Cellulose ethers which can be employed according to the invention are all known cellulose ethers which are hot water-coagulable and thus can be freed of salts with water at a temperature above their cloud point.

Preference is given to alkylcelluloses such as, for example, methyl-, ethyl- and propylcellulose, and mixed ethers thereof, such as, for example, hydroxyethyl-methyl-, hydroxypropyl methyl-, ethylhydroxyethyl- and ethyl methylcellulose.

There is no restriction on the degree of polymerization and the viscosity of the cellulose ethers to be employed. However, the starting materials of high viscosity used for the depolymerization are preferably cellulose ethers whose viscosity in 2% aqueous solution is more than 50 mPas.

The cellulose ethers particularly preferably employed according to the invention are obtained by a) alkalization of a cellulose with 0.5 to 10 mole equivalents of alkali, b) etherification of the resulting alkali cellulose with etherifying agents, c) reduction of the salt content to below 0.5% by weight by washing the cellulose ether with water at a temperature above the cloud point of the cellulose ether and removing the solid from the salt solution by centrifugation or filtration, so that the water content in the solid is in the range from 25 to 80% by weight, and d) simultaneous drying and grinding of the moist cellulose ether at temperatures in the range from 50 to 120° C. with the aid of a grinding/drying apparatus to result in a moisture content below 10% by weight.

A further advantage of the method of the invention compared with conventional methods is based on the fact that water pulps with a low α-cellulose content can also be used as starting pulps and, nevertheless, products with a high degree of whiteness result. This is because prior art methods normally result in colored products if the α-cellulose content is too low, which is the case in particular with water pulps of low quality. The color intensity increases as the viscosity of the depolymerized cellulose ether decreases. Although it is possible to minimize this problem in prior art methods by employing lintose pulps with a high α-cellulose content (>99%), the latter are costly and reduce the economic efficiency of the method. The pulps preferably used according to the invention have an α-cellulose content of from 90 to 99.9%, but particularly preferably a content of from 95 to 98%.

In a particularly preferred embodiment, cellulose ethers of very low viscosity, having Höppler viscosities measured at a concentration of 2% (absolutely dry) in water at 20° C., of ≦50 mPa·s are produced by the method of the invention.

Acids suitable for the hydrolytic decomposition are both mineral acids and organic acids, and mixtures thereof. However, mineral acids are preferred.

The mineral acids preferably employed are hydrochloric acid, sulfuric acid, nitric acid and phosphoric acid. However, it is also possible to use mixtures thereof.

Strong organic acids preferably employed are trifluoroacetic acid, acetic acid, formic acid, oxalic acid, phthalic acid, maleic acid and benzoic acid. It is, however, also possible to use mixtures thereof.

The amount of acid employed is preferably in the range from 0.01 to 2% by weight of pure acid based on the amount of cellulose ether employed. However, less than 1% by weight of acid is particularly preferably employed, and in particular less than 0.5% by weight of acid is employed. Acids with a pKa of <5.0 are preferably employed.

The exposure of the cellulose ether to the gaseous acid or the spraying with the acid solution preferably takes place at temperatures in the range from 20 to 120° C.

Oxidizing agents preferably employed are hydrogen peroxide and salts thereof, other peroxo compounds such as, for example, sodium peroxosulfate, ozone, perborates (also in combination with activators such as, for example, TAED), sodium chlorite, halogens, halogen oxides and other compounds used for bleaching. Hydrogen peroxide (H₂O₂) and ozone (O₃) are particularly preferred.

This is because if hydrogen peroxide is used as oxidizing agent to decompose it during the reaction without residues to water and oxygen. No other byproducts restricting the possible uses of the depolymerized cellulose ethers are formed. This is particularly important because the products are employed on a large scale in the drugs and foods sectors. Similar considerations apply to ozone in relation to the freedom from residues.

The oxidizing agents are preferably employed in amounts of from 0.01 to 3% by weight, particularly preferably from 0.2 to 1.5% by weight and in particular from 0.5 to 1.0% by weight, based on the cellulose ether.

The acid-catalyzed, hydrolytic oxidative decomposition of the invention is preferably carried out at temperatures in the range from 50 to 120° C. Temperatures in the range from 60 to 110° C. are particularly preferred.

The acid-catalyzed hydrolytic oxidative decomposition of the invention is preferably carried out under pressures in the range from 100 to 1030 mbar. Pressures in the range from 950 to 1030 mbar are particularly preferred.

Aqueous solutions of decomposed cellulose ethers generally have weakly acidic pH values owing to the generation of acidic groups on the basic cellulose ether framework. The pH of such solutions can be adjusted to a substantially neutral pH of 5.5 to 8.0 by admixing at least one basic salt, such as, for example, sodium carbonate or sodium bicarbonate, after the depolymerization. The at least one basic salt is preferably added as powder for this purpose, specifically in amounts of from 0.1 to 2.0, particularly preferably from 0.5 to 1.0, mole equivalents based on the amount of acid employed.

The viscosity of the resulting products can be adjusted essentially via the amounts of acid and oxidizing agent employed, the reaction time and the reaction temperature and is very reproducible.

The invention is explained in more detail below by means of exemplary embodiments without being restricted thereto, however.

The viscosities of the cellulose ethers produced in the examples are, unless otherwise indicated, measured in aqueous solution (2.0% strength based on the pure cellulose ether, at 20° C.) using a Höppler falling ball viscometer supplied by Haake.

The stated amounts of acid relate to % by weight pure HCl based on the amount of cellulose ether employed. The stated amounts of oxidizing agent (H₂O₂) likewise relate to % by weight pure H₂O₂ based on the amount of cellulose ether employed.

### EXAMPLES

### Example 1

Production of a cellulose ether of high viscosity 2.7 kg of a water pulp with a moisture content of about 3% by weight were introduced into 11.5 kg of dimethyl glycol under a nitrogen atmosphere in a reactor with a horizontal mixer shaft, and 0.23 kg of water and 1.87 kg of a concentrated sodium hydroxide solution (49.6% strength) were added. After 30 minutes, 0.56 kg of propylene oxide was added, and the mixture was heated to 80° C. and kept at this temperature for 60 minutes. A further 4.3 kg of 49.6% strength sodium hydroxide solution and 3.67 kg of methyl chloride were then added, and the mixture was heated to 100° C. and reacted at this temperature for 60 minutes. After the reaction was complete, the dimethyl glycol was distilled out under reduced pressure, and the crude product was washed with several portions of boiling water (total 100 kg), and separated from the slurry in each case. The residual moisture content after removal of the solid from the slurry was about 55 to 65% by weight, the residual salt content after the last washing step was 0.1% by weight. The material obtained in this way was ground and simultaneously dried in a Pallman PPSR mill which had been preheated to 80° C. to result in a fine-particle cellulose ether powder with a residual moisture content of about 1 to 3% by weight. The OCH₃ content was 29.7%, the OC₃H₈ content was 10.2% and the viscosity was 2 600 mPa·s measured on a 1.9% strength aqueous solution.

Depolymerization of the cellulose ether of high viscosity of example 1 to cellulose ethers of very low viscosity:

### Comparative example 2a

100 g of the cellulose ether powder from example 1 were sprayed with 0.25% HCl in the form of an aqueous solution so that, taking account of the water already present, the total water content of the system was 5.0% by weight. The material was transferred into a glass container and kept in continuous agitation for 2 hours at an oil bath temperature of 110° C. After the depolymerization, 0.7 times the molar equivalent amount of sodium carbonate, based on the amount of HCl added, was added and mixing was continued for a few minutes. A cellulose ether of very low viscosity having the characteristic indicated in table 1 results.

### Example 2b

100 g of the cellulose ether powder from example 1 were sprayed with 0.25% HCl in the form of an aqueous solution, and then with a dilute H₂O₂ solution (0.7 g of pure H₂O₂), so that, taking account of the water already present, the total water content of the system was 5.0% by weight. The material was transferred into a glass container and kept in continuous agitation for 2 hours at an oil bath temperature of 110C. After the depolymerization, 0.7 times the molar equivalent amount of sodium carbonate, based on the amount of HCl added, was added and mixing was continued for a few minutes.

A cellulose ether of very low viscosity having the characteristic indicated in table 1 results.

### Comparative example 3a

100 g of the cellulose ether powder from example 1 were sprayed with 0.5% HCl in the form of an aqueous solution so that, taking account of the water already present, the total water content of the system was 5.0% by weight. The material was transferred into a glass container and kept in continuous agitation for 3 hours at an oil bath temperature of 100° C. After the depolymerization, 0.7 times the molar equivalent amount of sodium carbonate, based on the amount of HCl added, was added and mixing was continued for a few minutes.

A cellulose ether of very low viscosity having the characteristic indicated in table 1 results.

### Example 3b

100 g of the cellulose ether powder from example 1 were sprayed with 0.5% HCl in the form of an aqueous solution, and then with a dilute H₂O₂ solution (1.05 g of pure H₂O₂), so that, taking account of the water already present, the total water content of the system was 5.0% by weight. The material was transferred into a glass container and kept in continuous agitation for 3 hours at an oil bath temperature of 110° C. After the depolymerization, 0.7 times the molar equivalent amount of sodium carbonate, based on the amount of HCl added, was added and mixing was continued for a few minutes.

A cellulose ether of very low viscosity having the characteristic indicated in table 1 results.

TABLE 1

| | HCl | H₂O₂ | | Degree of | |
|---|---|---|---|---|---|
| Ex. | (pure) | (pure) | Final viscosity | whiteness | Color of |
| No. | [% by wt.] | [% by wt.] | 2.0% [mPa · s] | of powder¹⁾ | solution²⁾ |
| 2a | 0.25 | — | 35 | 77 | 0.09 |
| 2b | 0.25 | 0.7 | 35 | 83 | 0.04 |
| 3a | 0.5 | — | 3 | 60 | 0.24 |
| 3b | 0.5 | 1.05 | 731 | | 0.09 |
| Determination methods: | | | | | |
| ¹⁾Degree of whiteness of powder: | | | | | |
| The basis for the measurement is DIN 5033; measurement with Color Tester LFM1 from Dr. Lange comparing with enamel white standard (setting: 82.7% reflectance) by measuring the reflectance at defined wavelength, blue filter (447 nm), normal light C; reproducibility ± 0.5. | | | | | |
| Because of the color-lightening effect when the particle size distribution is shifted to smaller values, direct comparison in relation to the degree of whiteness is permissible only for products with comparable particle size distributions. | | | | | |
| ²⁾Color of the solution: | | | | | |
| Measurement with CADAS 100 UV/VIS spectrophotometer from Dr. Lange, 2.0% by weight solution of the cellulose ether in water, cuvette 2 cm thick, by measuring the extinction at 415 and 578 nm (20° C.) and forming the difference. A larger value means a yellower solution. The color of the solution does not depend on the particle size distribution of the powder. However, it becomes more intense as the depolymerization of the material increases. For this reason, only cellulose ethers | | | | | |
| # of comparable viscosity should be compared with one another at the same concentration. | | | | | |

What is claimed is:

1. A method for the depolymerization of cellulose ethers by acid oxidative decomposition, comprising forming a reaction mixture by exposing ground and dried cellulose ether to gaseous acid or spraying ground and dried cellulose ether with an acid solution, then bringing the exposed or sprayed cellulose ether into contact with an oxidizing agent or an oxidizing agent solution, depolymerizing the cellulose ether at temperatures in the range from 50 to 120° C. over a period in the range from 0.01 to 10 hours, and subsequently neutralizing the acid by adding a base, wherein the water content of the reaction mixture does not exceed 10% by weight during the depolymerization.

2. The method as claimed in claim 1, wherein the cellulose ether comprises methyl-, ethyl-, propyl-, hydroxyethylmethyl-, hydroxypropylmethyl-, ethylhydroxyethyl- or ethylmethylcellulose.

3. The method as claimed in claim 1 wherein the cellulose ether is obtained by the steps of:
a) alkalization of a cellulose with 0.5 to 10 mole equivalents of alkali,
b) etherification of the resulting alkali cellulose with etherifying agents,
c) reduction of salt content to below 0.5% by weight by washing the cellulose ether with water at a temperature above the cloud point of the cellulose ether and removing solids by centrifugation or filtration, so that the water content in the solids is in the range from 25 to 80% by weight, and
d) simultaneous drying and grinding of the washed cellulose ether while moist at temperatures in the range from 50 to 120° C. with the aid of a grinding/drying apparatus to result in a moisture content below 10% by weight.

4. The method as claimed in claim 3, wherein step a) comprises the alkalization of a cellulose having a α-cellulose content of from 90 to 99.9%.

5. The method as claimed in claim 1, wherein the depolymerized cellulose ether has a Hoeppler viscosity measured at a concentration of 2.0% (absolutely dry) in water at 20° C. of ≦50 mPa·s.

6. The method as claimed in claim 1, wherein the acid comprises a mineral acid and/or organic acid.

7. The method as claimed in claim 6, wherein the mineral acid comprises hydrochloric, sulfuric, nitric and/or phosphoric acid.

8. The method as claimed in claim 6, wherein the organic acid comprises trifluoroacetic acid, acetic acid, formic acid, oxalic acid, phthalic acid, maleic acid, and/or benzoic acid.

9. The method as claimed in claim 1, wherein the acid is present in an amount in the range from 0.01 to 2% by weight of pure acid, based on the total amount of cellulose ether.

10. The method as claimed in claim 1, wherein the oxidizing agent comprises peroxo compounds, ozone, perborates, sodium chlorite, halogens and/or halogen oxides.

11. The method as claimed in claim 10, wherein the oxidizing agent comprises hydrogen peroxide.

12. The method as claimed in claim 10, wherein the oxidizing agent comprises ozone.

13. The method as claimed in claim 1, wherein the oxidizing agent is present in an amount of from 0.01 to 3% by weight based on the cellulose ether.

14. The method as claimed in claim 1, wherein the acid-catalyzed, hydrolytic oxidative decomposition is carried out at temperatures in the range from 50 to 120° C.

15. The method as claimed in claim 1, wherein the acid-catalyzed, hydrolytic oxidative decomposition is carried out under pressures in the range from 100 to 1030 mbar.

16. The method as claimed in claim 1, wherein 0.1 to 2.0 mole equivalents of at least one basic salt, based on the amount of acid employed, are added after depolymerization.

17. The method as claimed in claim 16, wherein the basic salt comprises sodium carbonate and/or sodium bicarbonate.

18. A method for depolymerizing cellulose ethers by acid oxidative decomposition, comprising the steps of:
a) exposing a cellulose ether to a gaseous acid or an acid solution;
b) contacting the exposed cellulose ether with an oxidizing agent or an oxidizing agent solution such that the cellulose ether is depolymerized at a temperature of from about 50 to about 120° C. over a period of time ranging from 0.01 to 10 hours; and then
c) neutralizing the acid by adding a base;
wherein the water content of the reaction mixture does not exceed 10% by weight during depolymerization.

19. The method of claim 18 wherein the cellulose ether of step (a) is a dried and ground cellulose ether.

20. The method of claim 19 wherein the cellulose ether is obtained by a process comprising the steps of:
a) alkalizing a cellulose with about 0.5 to about 10 mole equivalents of alkali;
b) etherifying the resulting alkali cellulose with etherifying agents to thereby form a cellulose ether;
c) reducing salt content to below about 0.5% by weight by washing the cellulose ether with water at a temperature above the cloud point of the cellulose ether, and removing solids by centrifugation or filtration, so that the water content in the solids is in the range from about 25 to about 80% by weight, and
d) simultaneous drying and grinding of the washed cellulose ether at temperatures in the range from about 50 to about 120° C. with the aid of a grinding/drying apparatus to result in a cellulose ether having a moisture content below about 10% by weight.
